(19) United States
(12) Patent Application Publication
(10) Pub. No.: US 2023/0220434 A1
(43) Pub. Date: Jul. 13, 2023

(54) COMPOSISTIONS AND METHODS FOR CRISPR ENABLED DNA SYNTHESIS

(71) Applicant: Duke University, Durham, NC (US)

(72) Inventors: Michael David LYNCH, Durham, NC (US); Eirik Adim MOREB, Durham, NC (US); Tian YANG, Durham, NC (US)

(73) Assignee: Duke University, Durham, NC (US)

(21) Appl. No.: 17/758,480

(22) PCT Filed: Jan. 8, 2021
(86) PCT No.: PCT/US2021/012607

§ 371 Date: Jul. 7, 2022

Related U.S. Application Data
(60) Provisional application No. 62/958,798, filed on Jan. 9, 2020.

(51) Int. Cl. C12P 19/34; C12N 15/11; C12N 9/22; C12Q 1/6844

(52) U.S. Cl. CPC C12P 19/34; C12N 15/11; C12N 9/22; C12Q 1/6844; C12N 2310/20

(57) ABSTRACT

Methods for CRISPR Enabled DNA Synthesis and compositions arising from the methods are provided. The methods may include ligation of partially single stranded DNA donor and acceptor oligonucleotides that are covalently linked to a subsequence of the target DNA to be sequenced followed by cleavage of the ligated product. In this manner the donor and acceptor oligonucleotides shuttle a growing subsequence of the target DNA with each cycle. A mutant Cpfl nuclease is missing non-specific ssDNA nuclease activity may be used for cleavage of the ligation product. Fourteen ligation/cleavage cycles can result in synthesis of ssDNA of greater than 10,000 bp in length.

### CROSS-REFERENCE TO RELATED APPLICATIONS

This application claims priority to U.S. Provisional Patent Application No. 62/958,798, filed Jan. 9, 2020, which is incorporated by reference herein in its entirety.

### STATEMENT AS TO FEDERALLY SPONSORED RESEARCH

This invention was made with Government support under Federal Grant no EE0007563 awarded by the Department of Energy (DOE). The Federal Government has certain rights to this invention.

### REFERENCE TO A SEQUENCE LISTING

The instant application contains a Sequence Listing which has been filed electronically in ASCII format as 47381-45_ST25.txt created on Jan. 6, 2021 and is 33837 bytes in size and is hereby incorporated by reference in its entirety.

### BACKGROUND

According to BCC Research, the current synthetic biology market will soon exceed $18 Billion USD annually. This market growth is in large part driven by key advances in technologies to both read and write DNA. The market for DNA or gene synthesis products alone is expected to exceed $7 Billion USO by 2024. The cost of synthesis has lagged significantly behind the reductions seen in the cost of DNA sequencing and on a per base pair level synthesis is still 5 orders of magnitude higher than that of DNA sequencing. The cost of DNA synthesis is still a major limiting factor in the field of synthetic biology.

At current best prices for DNA synthesis, even the synthesis of a relatively simple bacterial genomes, such as E. coli (˜5 Mbp) can be very costly. For the field of synthetic biology to realize its true potential, the cost of writing DNA needs to be reduced by at least 1000-fold to make DNA synthesis at the genome scale a feasible tool for routine systematic experimentation even in academic labs.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Toward this goal, we describe a next generation DNA synthesis technology “CEDS” or CRISPR Enabled DNA Synthesis. CEDS, has the potential to overcome many of the challenges associated with current methods of DNA synthesis and as a result also has the potential to enable extremely low costs for DNA synthesis and assembly. Traditional methodologies all still rely on the chemical synthesis of oligonucleotides, and the use of DNAs double stranded nature and enzymes to build larger dsDNA fragments. A key limitation in this methodology is the requirement for longer oligonucleotides, oftentimes in DNA synthesis from 100 bp to 200 bp, which are chemically synthesized (1 bp at a time). Synthesis of these oligonucleotides is expensive and subject to key yield limitations which are both a function of coupling efficiency. In addition, new oligonucleotides are required for each new synthesis project. The CEDs approach overcomes many of these challenges by enabling exponential single stranded DNA growth, for example 20 bp to 40 bp to 80 bp to 160 bp, etc. This exponential growth enables DNA fragments of up to 10 kilobases in less than 15 cycles reducing cycle number and compounding errors associated with oligo building technologies. In addition, as larger fragments are assembled as ssDNA and do not rely on hybridization of dsDNA for synthesis. Thus many issues currently limiting DNA synthesis methods such as secondary structures, and mis-hybridization will be minimized in the CEDs approach. Finally, the CEDs approach only requires a limited set of oligonucleotide sequences which can be purchased in bulk at high quality and reused for all synthesis projects.

Thus, herein described, in part, is a DNA synthesis methodology reliant on CRISPR nucleases, “CEDS”, or CRISPR Enabled DNA Synthesis, and compositions arising from the methods. In some aspects, the methods comprise the ligation of ssDNA DNA with terminal stem loop handles and the cleavage of these handles with a guide RNA targeted mutant Cpfl nuclease, where the mutant Cpfl nuclease is missing non-specific ssDNA nuclease activity. In other aspects, these steps are performed cyclically enabling exponential growth of linear ssDNA, from a limited set of common oligo precursors and without the need for any polymerases or template driven synthesis. In some aspects, only 14 cycles can lead to the synthesis of ssDNA of greater than 10,000 bp in length, and common smaller fragments can be used for the synthesis of multiple constructs in parallel.

In some aspects, the invention described a donor oligonucleotide having the following properties: a partially double stranded sequence formed by a hairpin loop; at least a six nucleotide base overhang at the 5′ end of the oligonucleotide; a blocked 3′ terminus; a sequence that is a protospacer adjacent motif, a sequence that is a RNA guided nuclease binding site; and a nuclease cleavage site at least 1 base from the 5′terminus of the oligonucleotide.

In some aspects, an extended donor oligonucleotide that has, at the 5′ terminus at least one nucleotide or a subsequence, N, of a target DNA sequence to be synthesized.

Similarly, in some aspects, the invention describes an acceptor oligonucleotide having the following properties: a partially double stranded sequence formed by a hairpin loop; at least a one nucleotide base overhang at the 3′ terminus of the oligonucleotide; a sequence that is a protospacer adjacent motif, a sequence that is a RNA guided nuclease binding site; and a nuclease cleavage site at least one base from the 3′ terminus of the oligonucleotide.

In some aspects, the acceptor oligonucleotide becomes an extended acceptor oligonucleotide when the oligonucleotide is covalently bound at the 3′ terminus to at least one nucleotide or subsequence, N, of a target DNA sequence to be synthesized.

In some aspects, the invention comprises a plurality of donor oligonucleotides, extended donor oligonucleotides, acceptor oligonucleotides or extended acceptor oligonucleotides, each with a unique nucleotide or nucleotide subsequence, N, of the target DNA to be synthesized. Any of these oligonucleotides may be complexed with a class II CRISPR/Cas Cpfl nuclease and a gRNA at the protospacer adjacent motif and nuclease binding site of the oligonucleotide. Any of these complexes may further be modified at any site with a purification tag or marker.

In some aspects, the invention provides a method of synthesizing a single stranded target DNA. The method includes the steps of: providing a plurality of donor and acceptor oligonucleotide, determining a starting point and order of addition of nucleotides necessary to form a complete target single stranded DNA sequence. Then performing repeated cycles of ligation of a 5′ terminus of a donor oligonucleotide comprising N, a nucleotide or nucleotide subsequence to the 3′ terminus of an acceptor oligonucleotide to create a ligated product; followed by contacting the ligated product with a guide RNA directed nuclease, to cleave the donor oligonucleotide leaving the N originating from the donor nucleotide covalently linked to the 3′ terminus of the acceptor nucleotide and repeating the cycle with a new donor oligonucleotide. The method produces a single stranded DNA product in a few steps that may be subjected to PCRT to produce larger volumes of a double stranded target DNA.

Importantly in some aspects, the guide RNA directed nuclease is a CRISPR nuclease lacking non-specific ssDNA nuclease activity.

### BRIEF DESCRIPTION OF THE DRAWINGS

The novel features of the invention are set forth with particularity in the appended claims. A better understanding of the features and advantages of the present invention may be obtained by reference to the following detailed description that sets forth illustrative aspects in which the principles of the invention are utilized, and the accompanying drawings of which:

FIG. 1A-D is a schematic showing an overview of CEDs in accordance with one aspect of the present disclosure.

FIG. 2A-E is a schematic and graph showing Cpfl-mediated cleavage during CEDS in accordance with one aspect of the present disclosure.

FIG. 3A-B is a schematic showing the processing/cleavage of the acceptor oligonucleotide in accordance with one aspect of the present disclosure. A) assay for cleavage reliant on a molecular beacons, and B) ligation and sequencing of cleaved acceptor oligonucleotides to confirm cleavage.

FIG. 4A-C is a schematic showing automated CEDS in accordance with one aspect of the present disclosure.

FIG. 5 is a graph showing gRNA binding to target DNA precludes molecular beacon binding in accordance with one aspect of the present disclosure.

### DETAILED DESCRIPTION

For the purposes of promoting an understanding of the principles of the present disclosure, reference will now be made to preferred aspects and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### 1. Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of’ (and grammatical variants) is to be interpreted as encompassing the recited materials or steps “and those that do not materially affect the basic and novel characteristic(s)” of the claimed invention. Thus, the term “consisting essentially of’ as used herein should not be interpreted as equivalent to “comprising.”

Moreover, the present disclosure also contemplates that in some aspects, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered expressly stated in this disclosure. Unless otherwise defined, all technical terms used herein have the same meaning as commonly understood by one of ordinary skill in the art to which this disclosure belongs.

PT represents a purification tag at or near the 5′ terminus of a donor oligonucleotide, acceptor oligonucleotide, or any extended donor and/or acceptor nucleotide (that is a donor or acceptor oligonucleotide contiguous with a subsequence of the nucleic acid to be synthesized). In some cases, this purification tag may be a magnetic bead covalently linked with the donor and/or acceptor oligonucleotide. The bead and/or tag may also be covalently linked to a gRNA or enzyme that complexes with the donor and/or acceptor oligonucleotide. It is appreciated though that any purification tag at any location within or attached to the donor and/or acceptor oligonucleotide can be encompassed as a purification tag (PT). Any affinity tag such as a fluorescent affinity tag or nucleotide or a streptavidin/biotin system, or other affinity ligand may be used. It may be appreciated that a purification tag may be added to any oligonucleotides useful for single stranded polynucleotide synthesis. The PT of the acceptor oligonucleotide and the donor oligonucleotide may be the same or different.

PAM represent a protospacer adjacent motif. PS represents a protospacer sequence. Protospacer sequences are a class of sequences recognized by enzymes of the CRISPR system. CS represents the site of cleavage by an endonuclease. Generally, the cleavage site is determined by the binding of an endonuclease to the double stranded recognition substrate in a polynucleotide such the hairpin loop of a donor or acceptor oligonucleotide.

N is a term applicable to a contiguous nucleotide sequence of any length. The term may be as small as one nucleotide or many contiguous nucleotides. The term contiguous describes more than one nucleotide covalently liked to each other and immediately adjacent to each other. The term N may represent subsequences of different lengths.

The terms partially and completely complementary and partially and completely hybridize or hybrid are used to describe the interaction between any oligonucleotides, polynucleotides, subsequence, or nucleic acid fragments of any length that are at least partially complimentary. The purpose of providing complementary sequences is to obtain a double stranded sequence recognizable by an endonuclease. That is to say that the hybridization between two complementary sequences needs to be sufficient to form an endonuclease recognition site but may not need to be completely perfectly hybridized or complementary to each other. There may be gaps or partially single stranded segments within a double stranded recognition sequence, yet not impede binding and cleavage by an endonuclease. Of interest is the PAM site and the sequence of the protospacer closest to the PAM site. Preferably these sequences are fully complementary.

Any contiguous nucleotide sequence of a target polynucleotide is generally formed of nucleotides from the group consisting of: A, G, T, or C. Likewise, the donor and acceptor oligonucleotides are also generally formed of nucleotides A, G, T, or C. It is appreciated though that variants or structural equivalents or mimics or non-natural nucleotides may also be used in the oligonucleotides of the invention and in the target polynucleotide that is synthesized by the methods described. For example, uracil, inosine, isoguanine, xanthine (5-(2,2 diamino pyrimidine), 8-azaguanine, 5 or 6-azauridine, 6-azacytidine, 4-hydroxypyrazolopyrimidine, allopurinol, arabinosyl cytosine, azathioprine, aminoallyl nucleotide, 5-bromouracil, any isomer of any natural or non-natural nucleotide, thiouridine, queuosine, wyosine, methyl-substituted phenyl analogs, purine or pyrimide mimics may be used.

### 2. Summary of Compositions

In some aspects, the invention described a donor oligonucleotide having the following properties: a partially double stranded sequence formed by a hairpin loop; at least a six nucleotide base overhang at the 5′ end of the oligonucleotide; a blocked 3′ terminus; a sequence that is a protospacer adjacent motif, a sequence that is a RNA guided nuclease binding site; and a nuclease cleavage site at least 1 base from the 5′ terminus of the oligonucleotide. The oligonucleotide is characterized by a melting temperature greater than 65° C.

In some aspects, the donor oligonucleotide further has, at the 5′ terminus at least one nucleotide, N, of a target DNA sequence to be synthesized. This may be termed an extended donor oligonucleotide. N may be a single nucleotide of a discreet subsequence of the target DNA being synthesized.

In some aspects, the invention comprises a plurality of extended donor oligonucleotides, each with a unique 5′ terminus nucleotide or nucleotide subsequence, N, of a target DNA to be synthesized.

In some aspects, the donor oligonucleotide may be complexed with a class II CRISPR/Cas Cpfl nuclease and a gRNA at the protospacer adjacent motif and nuclease binding site of the oligonucleotide. In some aspects the donor oligonucleotide, guide RNA or nuclease are modified with a purification tag. In some aspects, the tag is biotinylation.

Similarly, in some aspects, the invention describes an acceptor oligonucleotide comprising: a partially double stranded sequence formed by a hairpin loop; at least a one nucleotide base overhang at the 3′ terminus of the oligonucleotide; a sequence that is a protospacer adjacent motif, a sequence that is a RNA guided nuclease binding site; and a nuclease cleavage site at least one base from the 3′ terminus of the oligonucleotide where the acceptor oligonucleotide is characterized by a melting temperature greater than 65° C.

In some aspects, the acceptor oligonucleotide further carries, covalently bound to the 3′ terminus, at least one nucleotide or subsequence, N, of a target DNA sequence to be synthesized. This may be termed an extended acceptor oligonucleotide.

In some aspects, a plurality of extended acceptor oligonucleotides each with a unique 3′ terminus nucleotide or nucleotide subsequence, N, of a target DNA to be synthesized is provided.

In some aspects, the acceptor oligonucleotide or extended acceptor oligonucleotide is complexed with a class II CRISPR/Cas Cpfl nuclease and a gRNA at the protospacer adjacent motif and nuclease binding site of the oligonucleotide. In some aspects, the acceptor oligonucleotide, guide RNA or nuclease are modified with a purification tag. In some aspects, the tag is a biotinylation tag.

It is appreciated that while the donor and acceptor oligonucleotides are described as partially double stranded and having a hairpin loop, sequences of the oligonucleotides that are complementary to each other (and thus capable of forming a double stranded structure) may be linked to each other by any covalent means.

### 3. Invention Summary Methods

In some aspects, the invention provides a method of synthesizing a single stranded target DNA. The method includes the steps of: providing a plurality of donor and acceptor oligonucleotides including: donor oligonucleotides, extended donor oligonucleotides each with unique nucleotide, or a subsequence of the target DNA sequence to be synthesized covalently bound to the 5′ terminus, acceptor oligonucleotides, and extended acceptor nucleotides, each with unique nucleotide, or subsequence of the target DNA sequence to be synthesized covalently bound to the 3′ terminus. And next determining a starting point and order of addition of nucleotides necessary to form a complete target single stranded DNA sequence to be synthesized.

In some aspects the method continues with a ligating of the 5′ terminus of a donor oligonucleotide comprising N, a nucleotide or nucleotide subsequence determined to be the starting point, to the 3′ terminus of an acceptor oligonucleotide to create a ligated product; followed by contacting the ligated product with a guide RNA directed nuclease, to cleave the donor oligonucleotide leaving the N originating from the donor nucleotide covalently linked to the 3′ terminus of the acceptor nucleotide, thus producing an extended acceptor oligonucleotide. In this manner the donor and acceptor oligonucleotides serve as shuttles to transfer back and forth an ever-growing single stranded synthetic DNA sequence target.

In some aspects the method continues with a step of purifying the extended acceptor oligonucleotide; contacting the extended acceptor oligonucleotide, containing N, with an additional donor oligonucleotide; and repeating ligating, cleaving and purifying steps repeatedly, extending the subsequence N with each cycle, to obtain in the final step a complete single stranded target DNA.

In some aspects, the guide RNA directed nuclease is a CRISPR nuclease lacking non-specific ssDNA nuclease activity. In some aspects, the CRISPR nuclease is a mutant of Cpfl nuclease having mutations Q1025G and E1028G. In some aspects, the guide RNA directed nuclease is that of SEQ ID NO: 1. In some aspects, the guide RNA directed nuclease is encoded by SEQ ID NO: 2.

In some aspects, the complete single stranded target DNA that is formed by these methods is amplified via a polymerase chain reaction producing double stranded DNA.

In some aspects the donor oligonucleotide, gRNA, or guide RNA directed nuclease contain a purification tag and the step of purifying an extended acceptor oligonucleotide comprises removal of a complex formed between the donor oligonucleotide, gRNA, and nuclease via the purification tag.

In some aspects, the method may be performed with multiple ligation steps between donor and acceptor oligonucleotides occur synchronously and as separate reactions so that multiple purified subsequences are available for ligation to each other to obtain the final target DNA sequence in an exponential manner.

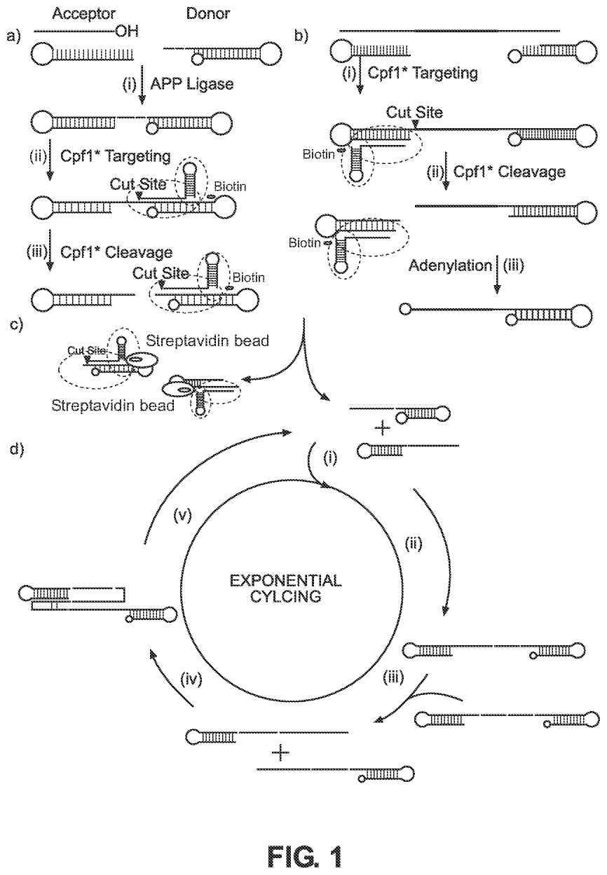

The CEDS process has the potential to overcome many of the challenges associated with current methods of DNA synthesis and as a result also has the potential to enable extremely low costs for DNA synthesis and assembly. As shown in FIG. 1, CEDS combines both linear and exponential single-stranded DNA synthesis to rapidly and efficiently build larger DNA fragments.

Referring again to FIG. 1, according to one aspect, the method, at minimum, begins with a limited set of 4 donor oligos, one for each nucleotide “A”, “T”, “C” and “G”. These hairpin structures are ligated to an acceptor oligonucleotide, and in some aspects, the donor and acceptor oligonucleotides have a hairpin structure. In one aspect, AppLigase, capable of non-specific ssDNA ligation, is used, wherein 5′ hydroxyl groups are first adenylated. A 3′ blocking group can be used to reduce non-specific ligations. In one aspect, the donor oligonucleotides contain a PAM and gRNA binding site specific for class II CRISPR/Cas Cpfl nuclease, which has been mutated to remove ssDNA nuclease activity, Cpfl*. The Cpfl* nuclease cuts the donor leaving the donated sequence ligated to the acceptor. The elongated acceptor can be ligated to new donors. In another aspect, as shown in FIG. 1B, donor oligonucleotides of extended length can be produced by cleaving the acceptor nucleotides from the ligated donor/acceptor pairs. In another aspect, the Cpfl* nuclease remains bound to its target after cleavage and can be removed from the reaction mixtures by pull down with magnetic beads, in this case with biotin on the gRNA (FIG. 1C). In yet another aspect and as shown in FIG. 1D, elongation of both acceptor and donor oligos can be used in a cycle enabling exponential growth of ssDNA.

### EXAMPLES

The following Examples are provided by way of illustration and not by way of limitation.

### Example 1. Ligation

Ligation of ssDNA (FIG. 1A) can be accomplished with existing enzymes. In one aspect, the enzyme comprises a thermostable AppLigase, an ATP dependent enzyme requiring 5′ pre-adenylated donors, which in the example case necessitated a two-step ligation, wherein donor oligonucleotides are first adenylated and then can be ligated to acceptor oligonucleotides with App Ligase. Mth RNA Ligase is used to convert phosphorylated 5′ DNA to App (Adenylated) DNA. Existing enzymes for ssDNA ligation were leverage and methods for CRISPR/Cas mediated cleavage of ligated products were be developed.

### Example 2. Cleavage of ssDNA at the 5′ End of Donor Oligonucleotides

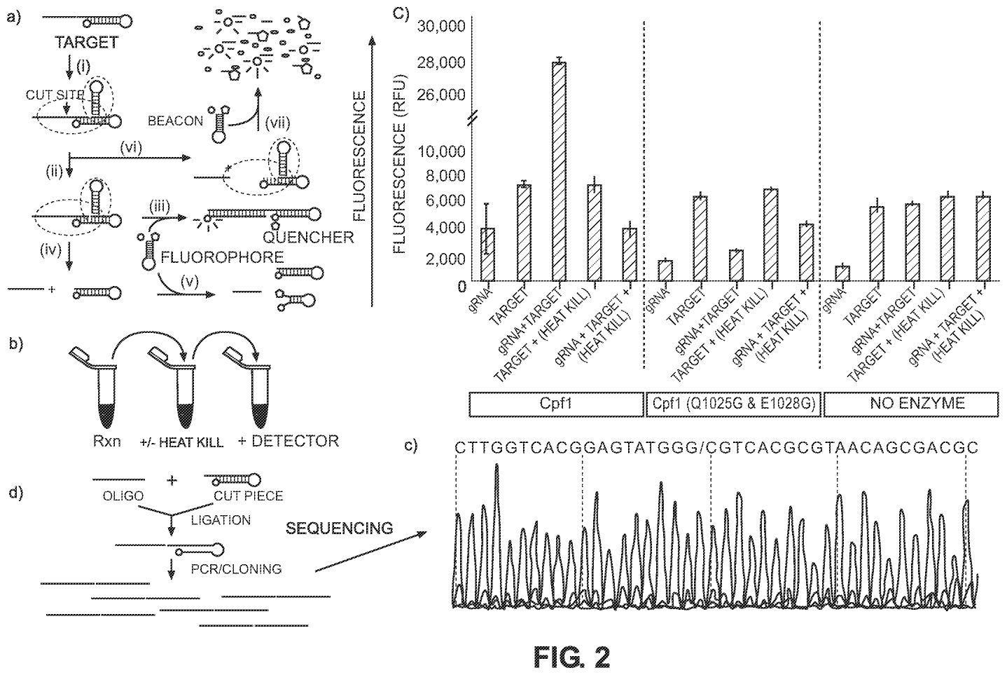

As can be seen in FIG. 1A, one of the key reactions in the CEDS process involves the gRNA targeted and Cpfl mediated cleavage of donor oligonucleotides leaving 5′ nucleotides as an extension on acceptor oligos. Cpfl, a class II CRISPR/Cas system can be used in this approach because it can cut 5′ of its recognition sequence removing the predefined gRNA target sequence from the growing DNA. To evaluate the 5′ donor cleavage step, we developed an assay reliant on a fluorescent molecular beacon as illustrated in FIG. 2.

This beacon specifically binds to a donor oligonucleotide, and when bound fluoresces. When the donor oligonucleotide is cleaved, the beacon can no longer bind and preferentially forms a hairpin which quenches fluorescence, as a result a decrease in fluorescence indicates donor DNA cleavage. A synthetic donor oligonucleotide was cleaved with Cpfl nuclease, and then the detector (molecular beacon) was added.

Wild type Cpfl, as well as other CRISPR/Cas nucleases contain non-specific nuclease activity which is activated once initial gRNA cleavage occurs. This is of course an unwanted reaction which degrades the linear DNA to be synthesized.

Referring specifically to FIG. 2, Cpfl mediated cleavage during CEDS is demonstrated. (A) A donor oligonucleotide is mixed with a gRNA Cpfl complex, which first binds (i) and then cuts the oligo (ii). In step (iii), in the event the donor oligo is not cut, once the molecular beacon is added it can hybridize to the oligo resulting in fluorescence. In step (iv), in the event the donor oligo is cut, the molecular-beacon preferentially forms a hairpin quenching fluorescence. In (v), in the case of wild type Cpfl enzyme with non-specific nuclease activity, after binding and cleavage occurs, nuclease activity will degrade any ssDNA present including the molecular beacon, releasing fluorophore, and greatly increasing fluorescence. (B) Cleavage reactions were carried with or without heat treatment prior to the addition of the detector (molecular beacon). C) Results of cleavage assays and appropriate controls. Wild type or mutant Cpfl (as well as no enzyme controls) were premixed with gRNA and used to cleave a donor oligonucleotide. (D) Cut donors, were ligated to synthetic oligos, amplified by PCR, and cloned into plasmids prior to sequencing. (E) A sample chromatogram of Sanger sequencing of clones confirming the correct cutting and ligation position. Ligation should occur between the highlighted G and C. Cutting successfully occurred 5′ of the C.

Fortunately, a mutant Cpfl nuclease Cpfl* (Cpfl(Q1025G,E1028G)) has been characterized, where non-specific nuclease activity has been abolished, enabling the CEDS process. As can be seen in FIG. 2, the use of wild type Cpfl, leads to an increase in fluorescence when the beacon is added, this is due to non-specific cleavage of the beacon itself, eliminating any quenching. Heat treatment of the reaction to kill Cpfl activity before adding the beacon, eliminates the increased fluorescence. In contrast Cpfl*, has the expected decrease in fluorescence on the addition of the beacon consistent with cleavage of the donor oligonucleotide and a loss of non-specific nuclease activity. Cleaved donor oligonucleotides were successfully adenylated and ligated to an acceptor oligo amplified by PCR and cloned (FIG. 2D), sequencing of these products (FIG. 2E) confirmed the correct cleavage and ligation position, and the success of cutting of the donor oligonucleotides.

### Example 3: Cleavage of ssDNA at the 3′ End of Acceptor Oligonucleotides

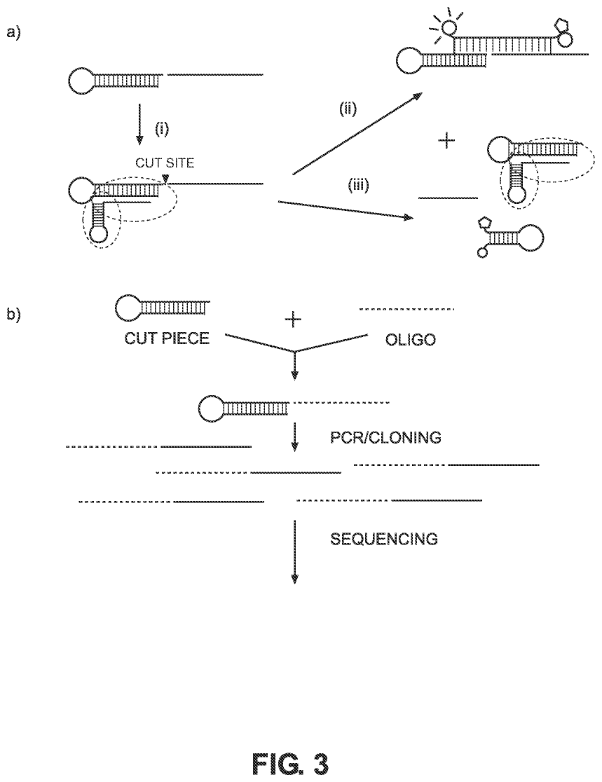

With the success of cutting the donor oligonucleotides we demonstrate the cleavage of the acceptor oligonucleotides. For the donor oligonucleotides, the disclosed method relies on cleavage of the non-target strand (NTS) 24 bp from the PAM site. However, the orientation of the target site on the acceptor oligo is such the target strand (TS) will instead be cleaved. TS cleavage occurs 19 bp from the PAM site on the same strand that the gRNA binds to. As illustrated in FIG. 3, we designed a hairpin at the 5′ end of the acceptor oligonucleotide and create a double stranded PAM site. As shown, this assay will again use a molecular beacon to confirm cleavage (FIG. 3A), followed by ligation and sequencing of the cleaved product (FIG. 3B).

### Example 4: gRNA Binding to Target DNA Precludes Molecular Beacon Binding

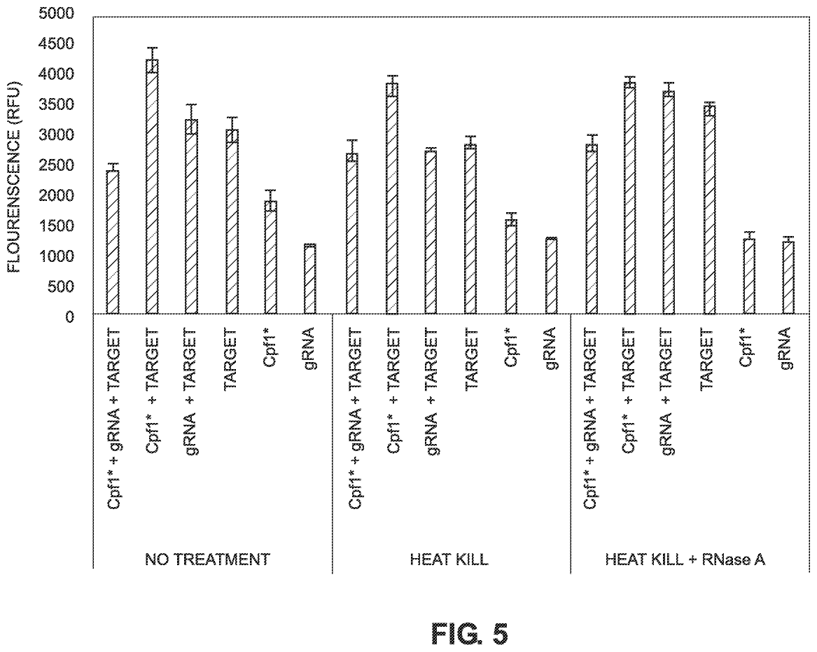

Referring to FIG. 5, gRNA binding to target DNA precludes molecular beacon binding in detail. In heat killed samples, the control, gRNA+Target, had the same low level of fluorescence as Cpfl*+gRNA+Target. This is due to the RNA binding to the target site and blocking the binding of the molecular beacon. To show this, RNAaseA was added and, as expected, the low level of fluorescence returned to uncut target levels.

### Example 5: Automated Cycling and DNA Synthesis

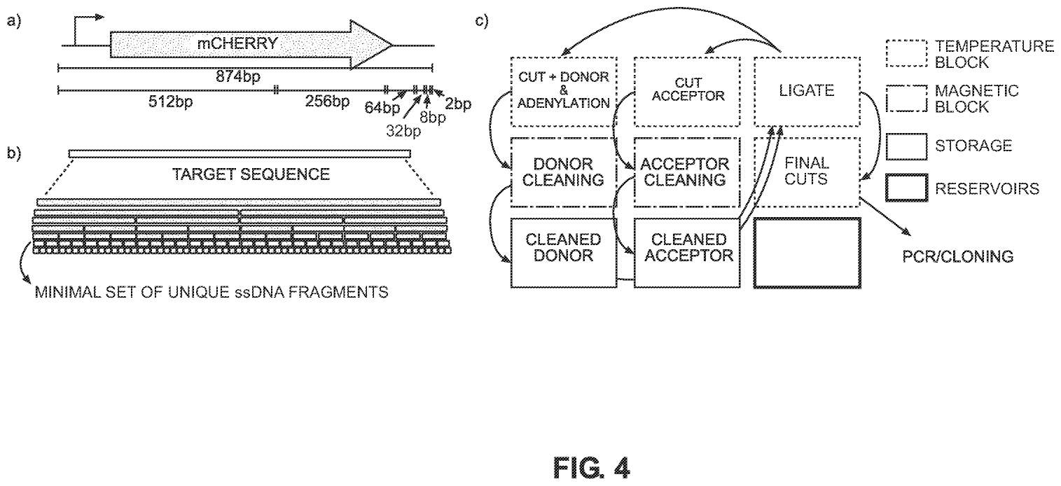

An important requirement for CEDS is the ability to capture and release linear DNA fragments, in a high throughput and iterative fashion. This is needed to be able to build desired DNA sequences from individual fragments in parallel. Toward this goal, an automated CEDS process using a liquid handler is illustrated in FIG. 4.

Referring specifically to FIG. 4, automated CEDS is described. (A) A target DNA sequence, in this case an mCherry expression construct is first split into subsequences which are amenable to exponential synthesis, in this case, an 874 bp DNA fragment is broken into a 512 bp and smaller exponential subsequences from 256 bp to 2 bp. (B) Computationally. the sequence of each subsequence is then split until single nucleotides are reached. At this point all unique fragment (red pieces) and repeat sequences (gray) are identified, creating a minimal set of unique sequences of each size. (C). Starting with 4 unique donors (A, T, C, and G), iterative rounds of adenylation/ligation and cleavage are performed, using 384 well plates, temperature blocks and magnetic plates. After each ligation, the reaction can potentially be split into two fractions, one where the donor is cut leading to an extended acceptor, and one where the acceptor is cut, leading to an extended donor. Cpfl* which stays bound to the donor and or acceptor oligos as well as the gRNA are removed from the reaction via a biotin covalently attached to the gRNA and a pull down with magnetic streptavidin beads. Cleaned extended acceptors and donors are then rearrayed for the next rounds of ligations. After the final ligations are complete, both ends are cleaved, and the ssDNA product amplified by PCR.

To reiterate, a target DNA sequence is first divided into pieces which are amenable to exponential synthesis, next computationally, the sequences of each piece are split into half until single nucleotides are reached. At this point all unique fragments and repeat sequences are identified, creating a minimal set of unique sequences of each size. Starting with 4 unique donor oligos (A, T, C, and G), iterative rounds of adenylation/ligation and cutting are then performed, using 384 well plates, temperature blocks and magnetic plates for purification. After each ligation the reaction can potentially be split into two factions, one where the donor is cut leading to an extended acceptor, and one where the acceptor is cut, leading to an extended donor (FIG. 4C). Cpfl* which stays bound to the donor and or acceptor oligos as well as gRNA are removed from the reaction via a biotin on the gRNA and a pull down with magnetic streptavidin beads. Cleaned extended acceptors and donors are then recombined for the next rounds of ligations. After the final ligations are complete, both ends are cleaved, and the ssDNA product amplified by PCR.

The CEDS approach overcomes many of these challenges by enabling exponential single stranded DNA growth, for example 2 bp to 4 bp to 8 bp to 16 bp, etc. This exponential growth enables DNA fragments of up to 10 kilobases in less than 14 cycles, reducing cycle number and compounding errors associated with oligo building technologies. In addition, as larger fragments are assembled as ssDNA and do not rely on hybridization of dsDNA for synthesis, we hypothesize that many issues currently limiting DNA synthesis methods such as secondary structures, and mis-hybridization will be minimized in the CEDs approach. Finally, the CEDS approach only requires a limited set of oligonucleotide sequences which can be purchased in bulk at high quality and reused for all synthesis projects, enabling large-scale multiplexed gene synthesis.

### Materials and Methods

### Cloning

6-His-MBP-TEV-FnCpfl was acquired from Addgene (Addgene ID 90094). Cpfl* was cloned via site directed mutagenesis using the oligos SEQ ID No: 4 and SEQ ID NO: 5. T4 PNK (NEB #M0201S), T4 Ligase (NEB #M0202S), and DpnI (NEB #R0l 76S) were used in the KLD reaction. Expression and Purification of Cpfl and Cpfl* Expression and purification of Cpfl and Cpfl*is adapted from. Cpfl and Cpfl* genes were expressed from a pET vector with a N-terminal 6×his-tag, followed by an MBP tag and a TEV cleavage site. 500 ml of low salt LB with 100 μg/ml ampicillin were inoculated with Rosetta(DE3) cells (Novagen) overnight culture containing each expression construct. The inoculated media was grown at 37° C. until the OD600 reached 0.6-1.0. A final concentration of 0.5 mM IPTG was added and the induction was allowed for 18 hours at 20° C. The culture was then harvested as 50 ml aliquots and frozen at −80° C. until purification. The cell pellet was resuspended in 10 ml of Lysis Buffer (20 mM HEPES, pH 7.5, 0.5M KCl, 25 mM imidazole, 0.1% Triton X-100) followed by 5 minutes of sonication (pulses with 10 sec on and 20 sec off) for cell disruption and the supernatant was applied to Ni2+-NT A-agarose resin in a drop column. The column was tumbled at 4° C. for 1 hour and then washed with 25 ml of Wash Buffer (20 mM HEPES, pH 7.5, 0.3M KCl, 25 mM imidazole) and then eluted with 4 ml of elution buffer (20 mM HEPES, pH 7.5, 0.15M KCl, 250 mM imidazole). The elution was then concentrated and exchanged to 500 μl of TEV Reaction Buffer (50 mM Tris, pH 7.5, 0.5 mM EDTA, 1 mM DTT) using centrifugal filter (Amicon) and supplemented with 200 units of TEV protease (NEB). The cleavage was allowed at 4° C. for 72 hours. The reaction was then applied to Ni²⁺-NTA-agarose resin to remove TEV protease and exchange to Storage Buffer (20 mM Tris, 0.15 M NaCl, 25% Glycerol) and stored at −20° C. until use.

### Single-Stranded DNA Cleavage Assay

Cleavage assays were performed using purified Cpfl or Cpfl*. 350 nM of Cpfl was used along with 700 nM of crRNA and 35 nM of 5′ Donor Oligonucleotide. Buffer 3.1 (NEB #7203S) was supplemented with 5 mM DTT. Total reaction volume was 10 μL. First, Cpfl was pre-incubated with crRNA for 10 min at room temperature. 5′ Donor Oligonucleotide was added, and the reaction was incubated at 37° C. for 15 min. Samples were then either left on ice or denatured at 95° C. for 10 min. To prevent RNA annealing to uncut ssDNA at the target site (FIG. 5), RNase A (GoldBio Cat #R-050-1) was added to the heat killed samples (final concentration of 100 μg/mL) while an equal volume of water was added to the non-heat treated samples. Samples were then incubated with the molecular beacon (SEQ ID NO: 15) for 10 min at room temperature and fluorescence was measured with excitation and emission at 492 nm and 535 nm, respectively.

### Adenylation

Adenylation was carried out using Mth RNA Ligase (NEB #E261 OS). The reaction was carried out by adding 10 μL of the heat killed Cpfl* reaction to the manufacturer's recommended protocol: 2 μL of Mth RNA Ligase, 2 μL of 10×5 DNA Adenylation Reaction Buffer, 2 μL of 1 mM ATP, and 4 μL of water for a total reaction volume of 20 μL. The reaction was incubated at 65° C. for 1 hour and then heat killed at 85° C. for 5 minutes.

### Ligation Assay

Ligations were carried out using Thermostable 5′ App RNA/DNA Ligase (NEB #M0319S). The adenylated Cpfl* reaction was ligated with an oligonucleotide (SEQ ID NO: 14) as described in FIG. 2. The 20 μL ligation reaction was carried out with 14 μL of adenylated Cpfl*, 1.2 μL of 5 uM SEQ ID NO: 14, 2 μL of NEBuffer 1, 2 μL of 50 mM MnCl₂, and 2 μL of Thermostable 5′ App RNA/DNA Ligase. The reaction was incubated at 65° C. overnight and then heat killed at 95° C. for 5 minutes. The ligated product was then PCR amplified with SEQ ID NO: 17 and SEQ ID NO: 18 using Econotaq DNA Polymerase (Lucigen #30035-1). The PCR product was purified and cloned via Golden Gate assembly using T4 DNA Ligase (NEB #M0202S) and Esp3i (NEB #R0734S) into SEQ ID NO: 19. Five clones were sent for Sanger sequencing at Genewiz (South Plainfield, N.J.) with sequencing primer SEQ ID NO: 20.

### Sequences

| Sequence | Function |
|---|---|
| MSIYQEFVNKYSLSKTLRFE | Cpf1* amino |
| LIPQGKTLENIKARGLILDD | Acid |
| EKRAKDYKKAKQIIDKYHQF | sequence |
| FIEEILSSVCISEDLLQNYS | |
| DVYFKLKKSDDDNLQKDFKS | |
| AKDTIKKQISEYIKDSEKFK | |
| NLFNQNLIDAKKGQESDLIL | |
| WLKQSKQNGIELFKANSDIT | |
| QIQEALEIIKSFKGWTTYFK | |
| GFHENRKNVYSSNDIPTSII | |
| YRIVDDNLPKFLENKAKYES | |
| LKDKAPEAINYEQIKKDLAE | |
| ELTFDIDYKTSEVNQRVFSL | |
| DEVFEIANFNNYLNQSGITK | |
| FNTIIGGKFVNGENTKRKGI | |
| NEYINLYSQQINDKTLKKYK | |
| MSVLFKQILSDTESKSFVID | |
| KLEQDSDVVTTMQSFYEQIA | |
| AFKTVEEKSIKETLSLLFDD | |
| LKAQKLDLSKIYFKNDKSLT | |
| DLSQQVFQDYSVIGTAVLEY | |
| ITQQIAPKNLDNPSKKEQEL | |
| IAKKTEKAKYLSLETIKLAL | |
| EEFNKHRDIDKQCRFEEILA | |
| NFAAIPMIFDEIAQNKDNLA | |
| QISIKYQNQGKKDLLQASAE | |
| DDVKAIKDLLDQTNNLLHKL | |
| KIFHISQSEDKANILDKDEH | |
| FYLVFEECYFELANIVPLYN | |
| KIRNYITQKPYSDEKFKLNF | |
| ENSTLANGWQKNKEPDNTAI | |
| LFIKDDKYYLGVMNKKNNKI | |
| FDDKAIKENKGEGYKKIVYK | |
| LLPGANKMLPKVFFSAKSIK | |
| FYNPSEDILRIRNHSTHTKN | |
| GSPQKGYEKFEFNIEDCRKF | |
| IDFYKQSISKHPEWKDFGFR | |
| FSDTQRYNSIDEFYREVENQ | |
| GYKLTFENISESYIDSWNQG | |
| KLYLFQIYNKDFSAYSKGRP | |
| NLHTLYWKALFDERNLQDVV | |
| YKLNGEAELFYRKQSIPKKI | |
| THPAKEAIANKNKDNPKKES | |
| VFEYDLIKDKRFTEDKFFFH | |
| CPITINFKSSGANKFNDEIN | |
| LLLKEKANDVHILSIDRGER | |
| HLAYYTLVDGKGNIIKQDTF | |
| NIIGNDRMKTNYHDKIMIEK | |
| DRDSARKDWKKINNIKEMKE | |
| GYLSQVVHEIAKLVIEYNAI | |
| WFEDLNFGFKRGRFKVEKQV | |
| YGKLGKMLIEKLNYLVFKDN | |
| EFDKTGGVLRAYQLTAPFET | |
| FKKMGKQTGIIYYVPAGFTS | |
| KICPVTGFVNQLYPKYESVS | |
| KSQEFFSKFDKICYNLDKGY | |
| FEFSFDYKNFGDKAAKGKWT | |
| IASFGSRLINFRNSDKNHNW | |
| DTREVYPTKELEKLLKDYSI | |
| EYGHGECIKAAICGESDKKF | |
| FAKLTSVLNTILQMRNSKTG | |
| TELDYLISPVADVNGNFFDS | |
| RQAPKNMPQDADANGAYHIG | |
| LKGLMLLGRIKNNQEGKKLN | |
| LVIKNEEYFEFVQNRNN | |
| (SEQ ID NO: 1) | |
| ATGAGCATCTACCAGGAGTT | Cpf1* DNA |
| CGTCAACAAGTATTCACTGA | sequence |
| GTAAGACACTGCGGTTCGAG | |
| CTGATCCCACAGGGCAAGAC | |
| ACTGGAGAACATCAAGGCCC | |
| GAGGCCTGATTCTGGACGAT | |
| GAGAAGCGGGCAAAAGACTA | |
| TAAGAAAGCCAAGCAGATCA | |
| TTGATAAATACCACCAGTTC | |
| TTTATCGAGGAAATTCTGAG | |
| CTCCGTGTGCATCAGTGAGG | |
| ATCTGCTGCAGAATTACTCA | |
| GACGTGTACTTCAAGCTGAA | |
| GAAGAGCGACGATGACAACC | |
| TGCAGAAGGACTTCAAGTCC | |
| GCCAAGGACACCATCAAGAA | |
| ACAGATTAGCGAGTACATCA | |
| AGGACTCCGAAAAGTTTAAA | |
| AATCTGTTCAACCAGAATCT | |
| GATCGATGCTAAGAAAGGCC | |
| AGGAGTCCGACCTGATCCTG | |
| TGGCTGAAACAGTCTAAGGA | |
| CAATGGGATTGAACTGTTCA | |
| AGGCTAACTCCGATATCACT | |
| GATATTGACGAGGCACTGGA | |
| AATCATCAAGAGCTTCAAGG | |
| GATGGACCACATACTTTAAA | |
| GGCTTCCACGAGAACCGCAA | |
| GAACGTGTACTCCAGCAACG | |
| ACATTCCTACCTCCATCATC | |
| TACCGAATCGTCGATGACAA | |
| TCTGCCAAAGTTCCTGGAGA | |
| ACAAGGCCAAATATGAATCT | |
| CTGAAGGACAAAGCTCCCGA | |
| GGCAATTAATTACGAACAGA | |
| TCAAGAAAGATCTGGCTGAG | |
| GAACTGACATTCGATATCGA | |
| CTATAAGACTAGCGAGGTGA | |
| ACCAGAGGGTCTTTTCCCTG | |
| GACGAGGTGTTTGAAATCGC | |
| CAATTTCAACAATTACCTGA | |
| ACCAGTCCGGCATTACTAAA | |
| TTCAATACCATCATTGGCGG | |
| GAAGTTTGTGAACGGGGAGA | |
| ATACCAAGCGCAAGGGAATT | |
| AACGAATACATCAATCTGTA | |
| TAGCCAGCAGATCAACGACA | |
| AAACTCTGAAGAAATACAAG | |
| ATGTCTGTGCTGTTCAAACA | |
| GATCCTGAGTGATACCGAGT | |
| CCAAGTCTTTTGTCATTGAT | |
| AAACTGGAAGATGACTCAGA | |
| CGTGGTCACTACCATGCAGA | |
| GCTTTTATGAGCAGATCGCC | |
| GCTTTCAAGACAGTGGAGGA | |
| AAAATCTATTAAGGAAACTC | |
| TGAGTCTGCTGTTCGATGAC | |
| CTGAAAGCCCAGAAGCTGGA | |
| CCTGAGTAAGATCTACTTCA | |
| AAAACGATAAGAGTCTGACA | |
| GACCTGTCACAGCAGGTGTT | |
| TGATGACTATTCCGTGATTG | |
| GGACCGCCGTCCTGGAGTAC | |
| ATTACACAGCAGATCGCTCC | |
| AAAGAACCTGGATAATCCCT | |
| CTAAGAAAGAGCAGGAACTG | |
| ATCGCTAAGAAAACCGAGAA | |
| GGCAAAATATCTGAGTCTGG | |
| AAACAATTAAGCTGGCACTG | |
| GAGGAGTTCAACAAGCACAG | |
| GGATATTGACAAACAGTGCC | |
| GCTTTGAGGAAATCCTGGCC | |
| AACTTCGCAGCCATCCCCAT | |
| GATTTTTGATGAGATCGCCC | |
| AGAACAAAGACAATCTGGCT | |
| CAGATCAGTATTAAGTACCA | |
| GAACCAGGGCAAGAAAGACC | |
| TGCTGCAGGCTTCAGCAGAA | |
| GATGACGTGAAAGCCATCAA | |
| GGATCTGCTGGACCAGACCA | |
| ACAATCTGCTGCACAAGCTG | |
| AAAATCTTCCATATTAGTCA | |
| GTCAGAGGATAAGGCTAATA | |
| TCCTGGATAAAGACGAACAC | |
| TTCTACCTGGTGTTCGAGGA | |
| ATGTTACTTCGAGCTGGCAA | |
| ACATTGTCCCCCTGTATAAC | |
| AAGATTAGGAACTACATCAC | |
| ACAGAAGCCTTACTCTGACG | |
| AGAAGTTTAAACTGAACTTC | |
| GAAAATAGTACCCTGGCCAA | |
| CGGGTGGGATAAGAACAAGG | |
| AGCCTGACAACACAGCTATC | |
| CTGTTCATCAAGGATGACAA | |
| GTACTATCTGGGAGTGATGA | |
| ATAAGAAAAACAATAAGATC | |
| TTCGATGACAAAGCCATTAA | |
| GGAGAACAAAGGGGAAGGAT | |
| ACAAGAAAATCGTGTATAAG | |
| CTGCTGCCCGGCGCAAATAA | |
| GATGCTGCCTAAGGTGTTCT | |
| TCAGCGCCAAGAGTATCAAA | |
| TTCTACAACCCATCCGAGGA | |
| CATCCTGCGGATTAGAAATC | |
| ACTCAACACATACTAAGAAC | |
| GGGAGCCCCCAGAAGGGATA | |
| TGAGAAATTTGAGTTCAACA | |
| TCGAGGATTGCAGGAAGTTT | |
| ATTGACTTCTACAAGCAGAG | |
| CATCTCCAAACACCCTGAAT | |
| GGAAGGATTTTGGCTTCCGG | |
| TTTTCCGACACACAGAGATA | |
| TAACTCTATCGACGAGTTCT | |
| ACCGCGAGGTGGAAAATCAG | |
| GGGTATAAGCTGACTTTTGA | |
| GAACATTTCTGAAAGTTACA | |
| TCGACAGCGTGGTCAATCAG | |
| GGAAAGCTGTACCTGTTCCA | |
| GATCTATAACAAAGATTTTT | |
| CAGCATACAGCAAGGGCAGA | |
| CCAAACCTGCATACACTGTA | |
| CTGGAAGGCCCTGTTCGATG | |
| AGAGGAATCTGCAGGACGTG | |
| GTCTATAAACTGAACGGAGA | |
| GGCCGAACTGTTTTACCGGA | |
| AGCAGTCTATTCCTAAGAAA | |
| ATCACTCACCCAGCTAAGGA | |
| GGCCATCGCTAACAAGAACA | |
| AGGACAATCCTAAGAAAGAG | |
| AGCGTGTTCGAATACGATCT | |
| GATTAAGGACAAGCGGTTCA | |
| CCGAAGATAAGTTCtttttc | |
| cattgtccaatcaccattaa | |
| cttcAAGTCAAGCGGCGCTA | |
| ACAAGTTCAACGACGAGATC | |
| AATCTGCTGCTGAAGGAAAA | |
| AGCAAACGATGTGCACATCC | |
| TGAGCATTGACCGAGGAGAG | |
| CGGCATCTGGCCTACTATAC | |
| CCTGGTGGATGGCAAAGGGA | |
| ATATCATTAAGCAGGATACA | |
| TTCAACATCATTGGCAATGA | |
| CCGGATGAAAACCAACTACC | |
| ACGATAAACTGGCTGCAATC | |
| GAGAAGGATAGAGACTCAGC | |
| TAGGAAGGACTGGAAGAAAA | |
| TCAACAACATTAAGGAGATG | |
| AAGGAAGGCTATCTGAGCCA | |
| GGTGGTCCATGAATTGCAAA | |
| GCTGGTCATCGAATACAATG | |
| CCATTGTGGTGTTCGAGGAT | |
| CTGAACTTCGGCTTTAAGAG | |
| GGGGCGCTTTAAGGTGGAAA | |
| AACAGGTCTATggcAAGCTg | |
| gcAAAATGCTGATCGAAAAG | |
| CTGAATTACCTGGTGTTTAA | |
| AGATAACGAGTTCGACAAGA | |
| CCGGAGGCGTCCTGAGAGCC | |
| TACCAGCTGACAGCTCCCTT | |
| TGAAACTTTCAAGAAAATGG | |
| GAAAACAGACAGGCATCATC | |
| TACTATGTGCCAGCCGGATT | |
| CACTTCCAAGATCTGCCCCG | |
| TGACCGGCTTTGTCAACCAC | |
| TGTACCCTAAATATGAGTCA | |
| GTGAGCAAGTCCCAGGAATT | |
| TTTCAGCAAGTTCGATAAGA | |
| TCTGTTATAATCTGGACAAG | |
| GGGTACTTCGAGTTTTCCTT | |
| CGATTACAAGAACTTCGGCG | |
| ACAAGGCCGCTAAGGGGAAA | |
| TGGACCATTGCCTCCTTCGG | |
| ATCTCGCCTGATCAACTTTC | |
| GAAATTCCGATAAAAACCAC | |
| AATTGGGACACTAGGGAGGT | |
| GTACCCAACCAAGGAGCTGG | |
| AAAAGCTGCTGAAAGACTAC | |
| TCTATCGAGTATGGACATGG | |
| CGAATGCATCAAGGCAGCCA | |
| TCTGTGGCGAGAGTGATAAG | |
| AAATTTTTCGCCAAGCTGAC | |
| CTCAGTGCTGAATACAATCC | |
| TGCAGATGCGGAACTCAAAG | |
| ACCGGGACAGAACTGGACTA | |
| TCTGATTAGCCCCGTGGCTG | |
| ATGTCAACGGAAACTTCTTC | |
| GACAGCAGACAGGCACCCAA | |
| AAATATGCCTCAGGATGCAG | |
| ACGCCAACGGGGCCTACCAC | |
| ATCGGGCTGAAGGGACTGAT | |
| GCTGCTGGGCCGGATCAAGA | |
| ACAATCAGGAGGGGAAGAAG | |
| CTGAACCTGGTCATTAAGAA | |
| CGAGGAATACTTCGAGTTTG | |
| TCCAGAATAGAAATAACTAA | |
| (SEQ ID NO: 2) | |
| ATGAGCATCTACCAGGAGTT | Cpf1 DNA |
| CGTCAACAAGTATTCACTGA | sequence |
| GTAAGACACTGCGGTTCGAG | |
| CTGATCCCACAGGGCAAGAC | |
| ACTGGAGAACATCAAGGCCC | |
| GAGGCCTGATTCTGGACGAT | |
| GAGAAGCGGGCAAAAGACTA | |
| TAAGAAAGCCAAGCAGATCA | |
| TTGATAAATACCACCAGTTC | |
| TTTATCGAGGAAATTCTGAG | |
| CTCCGTGTGCATCAGTGAGG | |
| ATCTGCTGCAGAATTACTCA | |
| GACGTGTACTTCAAGCTGAA | |
| GAAGAGCGACGATGACAACC | |
| TGCAGAAGGACTTCAAGTCC | |
| GCCAAGGACACCATCAAGAA | |
| ACAGATTAGCGAGTACATCA | |
| AGGACTCCGAAAAGTTTAAA | |
| AATCTGTTCAACCAGAATCT | |
| GATCGATGCTAAGAAAGGCC | |
| AGGAGTCCGACCTGATCCTG | |
| TGGCTGAAACAGTCTAAGGA | |
| CAATGGGATTGAACTGTTCA | |
| AGGCTAACTCCGATATCACT | |
| GATATTGACGAGGCACTGGA | |
| AATCATCAAGAGCTTCAAGG | |
| GATGGACCACATACTTTAAA | |
| GGCTTCCACGAGAACCGCAA | |
| GAACGTGTACTCCAGCAACG | |
| ACATTCCTACCTCCATCATC | |
| TACCGAATCGTCGATGACAA | |
| TCTGCCAAAGTTCCTGGAGA | |
| ACAAGGCCAAATATGAATCT | |
| CTGAAGGACAAAGCTCCCGA | |
| GGCAATTAATTACGAACAGA | |
| TCAAGAAAGATCTGGCTGAG | |
| GAACTGACATTCGATATCGA | |
| CTATAAGACTAGCGAGGTGA | |
| ACCAGAGGGTCTTTTCCCTG | |
| GACGAGGTGTTTGAAATCGC | |
| CAATTTCAACAATTACCTGA | |
| ACCAGTCCGGCATTACTAAA | |
| TTCAATACCATCATTGGCGG | |
| GAAGTTTGTGAACGGGGAGA | |
| ATACCAAGCGCAAGGGAATT | |
| AACGAATACATCAATCTGTA | |
| TAGCCAGCAGATCAACGACA | |
| AAACTCTGAAGAAATACAAG | |
| ATGTCTGTGCTGTTCAAACA | |
| GATCCTGAGTGATACCGAGT | |
| CCAAGTCTTTTGTCATTGAT | |
| AAACTGGAAGATGACTCAGA | |
| CGTGGTCACTACCATGCAGA | |
| GCTTTTATGAGCAGATCGCC | |
| GCTTTCAAGACAGTGGAGGA | |
| AAAATCTATTAAGGAAACTC | |
| TGAGTCTGCTGTTCGATGAC | |
| CTGAAAGCCCAAAGCGTGG | |
| ACCTGAGTAAGATCTACTTC | |
| AAAAACGATAAGAGTCTGAC | |
| AGACCTGTCACAGCAGGTGT | |
| TTGATGACTATTCCGTGATT | |
| GGGACCGCCGTCCTGGAGTA | |
| CATTACACAGCAGATCGCTC | |
| CAAAGAACCTGGATAATCCC | |
| TCTAAGAAAGAGCAGGAACT | |
| GATCGCTAAGAAAACCGAGA | |
| AGGCAAAATATCTGAGTCTG | |
| GAAACAATTAAGCTGGCACT | |
| GGAGGAGTTCAACAAGCACA | |
| GGGATATTGACAAACAGTGC | |
| CGCTTTGAGGAAATCCTGGC | |
| CAACTTCGCAGCCATCCCCA | |
| TGATTTTTGATGAGATCGCC | |
| CAGAACAAAGACAATCTGGC | |
| TCAGATCAGTATTAAGTACC | |
| AGAACCAGGGCAAGAAAGAC | |
| CTGCTGCAGGCTTCAGCAGA | |
| AGATGACGTGAAAGCCATCA | |
| AGGATCTGCTGGACCAGACC | |
| AACAATCTGCTGCACAAGCT | |
| GAAAATCTTCCATATTAGTC | |
| AGTCAGAGGATAAGGCTAAT | |
| ATCCTGGATAAAGACGAACA | |
| CTTCTACCTGGTGTTCGAGG | |
| AATGTTACTTCGAGCTGGCA | |
| AACATTGTCCCCCTGTATAA | |
| CAAGATTAGGAACTACATCA | |
| CACAGAAGCCTTACTCTGAC | |
| GAGAAGTTTAAACTGAACTT | |
| CGAAAATAGTACCCTGGCCA | |
| ACGGGTGGGATAAGAACAAG | |
| GAGCCTGACAACACAGCTAT | |
| CCTGTTCATCAAGGATGACA | |
| AGTACTATCTGGGAGTGATG | |
| AATAAGAAAAACAATAAGAT | |
| CTTCGATGACAAAGCCATTA | |
| AGGAGAACAAAGGGGAAGGA | |
| TACAAGAAAATCGTGTATAA | |
| GCTGCTGCCCGGCGCAAATA | |
| AGATGCTGCCTAAGGTGTTC | |
| TTCAGCGCCAAGAGTATCAA | |
| ATTCTACAACCCATCCGAGG | |
| ACATCCTGCGGATTAGAAAT | |
| CACTCAACACATACTAAGAA | |
| CGGGAGCCCCCAGAAGGGAT | |
| ATGAGAAATTTGAGTTCAAC | |
| ATCGAGGATTGCAGGAAGTT | |
| TATTGACTTCTAGGAAGGAT | |
| TTTGGCTTCCGGTTTTCCGA | |
| CACACAGAGATATAACTCTA | |
| TCGACGAGTTCTACCGCGAG | |
| GTGGAAAATCAGGGGTATAA | |
| GCTGACTTTTGAGAACATTT | |
| CTGAAAGTTACATCGACAGC | |
| GTGGTCAATCAGGGAAAGCT | |
| GTACCTGTTCCAGATCTATA | |
| ACAAAGATTTTTCAGCATAC | |
| AGCAAGGGCAGACCAAACCT | |
| GCATACACTGTACTGGAAGG | |
| CCCTGTTCGATGAGAGGAAT | |
| CTGCAGGACGTGGTCTATAA | |
| ACTGAACGGAGAGGCCGAAC | |
| TGTTTTACCGGAAGCAGTCT | |
| ATTCCTAAGAAAATCACTCA | |
| CCCAGCTAAGGAGGCCATCG | |
| CTAACAAGAACAAGGACAAT | |
| CCTAAGAAAGAGAGCGTGTT | |
| CGAATACGATCTGATTAAGG | |
| ACAAGCGGTTCACCGAAGAT | |
| AAGTTCTTTTTCCATTGTCC | |
| AATCACCATTAACTTCAAGT | |
| CAAGCGGCGCTAACAAGTTC | |
| AACGACGAGATCAATCTGCT | |
| GCTGAAGGAAAAAGCAAACG | |
| ATGTGCACATCCTGAGCATT | |
| GACCGAGGAGAGCGGCATCT | |
| GGCCTACTATACCCTGGTGG | |
| ATGGCAAAGGGAATATCATT | |
| AAGCAGGATACATTCAACAT | |
| CATTGGCAATGACCGGATGA | |
| AAACCAACTACCACGATAAA | |
| CTGGCTGCAATCGAGAAGGA | |
| TAGAGACTCAGCTAGGAAGG | |
| ACTGGAAGAAAATCAACAAC | |
| ATTAAGGAGATGAAGGAAGG | |
| CTATCTGAGCCAGGTGGTCC | |
| ATGAGATTGCAAAGCTGGTC | |
| ATCGAATACAATGCCATTGT | |
| GGTGTTCGAGGATCTGAAC | |
| TTCGGCTTTAAGAGGGGGCG | |
| CTTTAAGGTGGAAAAACAGG | |
| TCTATCAGAAGCTGGAGAAA | |
| ATGCTGATCGAAAAGCTGAA | |
| TTACCTGGTGTTTAAAGATA | |
| ACGAGTTCGACAAGACCGGA | |
| GGCGTCCTGAGAGCCTACCA | |
| GCTGACAGCTCCCTTTGAAA | |
| CTTTCAAGAAAATGGGAAAA | |
| CAGACAGGCATCATCTACTA | |
| TGTGCCAGCCGGATTCACTT | |
| CCAAGATCTGCCCCGTGACC | |
| GGCTTTGTCAACCAGCTGTA | |
| CCCTAAATATGAGTCAGTGA | |
| GCAAGTCCCAGGAATTTTTC | |
| AGCAAGTTCGATAAGATCTG | |
| TTATAATCTGGACAAGGGGT | |
| ACTTCGAGTTTTCCTTCGAT | |
| TACAAGAACTTCGGCGACAA | |
| GGCCGCTAAGGGGAAATGGA | |
| CCATTGCCTCCTTCGGATCT | |
| CGCCTGATCAACTTTCGAAA | |
| TTCCGATAAAAACCACAATT | |
| GGGACACTAGGGAGGTGTAC | |
| CCAACCAAGGAGCTGGAAAA | |
| GCTGCTGAAAGACTACTCTA | |
| TCGAGTATGGACATGGCGAA | |
| TGCATCAAGGCAGCCATCTG | |
| TGGCGAGAGTGATAAGAAAT | |
| TTTTCGCCAAGCTGACCTCA | |
| GTGCTGAATACAATCCTGCA | |
| GATGCGGAACTCAAAGACCG | |
| GGACAGAACTGGACTATCTG | |
| ATTAGCCCCGTGGCTGATGT | |
| CAACGGAAACTTCTTCGACA | |
| GCAGACAGGCACCCAAAAAT | |
| ATGCCTCAGGATGCAGACGC | |
| CAACGGGGCCTACCACATCG | |
| GGCTGAAGGGACTGATGCTG | |
| CTGGGCCGGATCAAGAACAA | |
| TCAGGAGGGGAAGAAGCTGA | |
| ACCTGGTCATTAAGAACGAG | |
| GAATACTTCGAGTTTGTCCA | |
| GAATAGAAATAAC | |
| (SEQ ID NO: 3) | |
| CTGGGCAAAATGCTGATCG | Forward |
| AAAAGCTGAA TTACCTGG | primer to |
| (SEQ ID NO: 4) | make Cpt1* |
| | from Cpf1 |
| CTTGCCATAGACCTGTTTTT | Reverse |
| CCACCTTAAA GC | primer to |
| (SEQ ID NO: 5) | make Cpf1 • |
| | from Cpf1 |
| AAGGAATGGTGCATGCAAGG | Cpf1 |
| (SEQ ID NO: 6) | sequencing |
| | primer |
| CGAATCCGCCTAAAACCTGG | Cpf1 |
| (SEQ ID NO: 7) | sequencing |
| | primer |
| ATTAATGCCGCATCAGGTCG | Cpf1 |
| (SEQ ID NO: 8) | sequencing |
| | primer |
| TCCTGGAGAACAAGGCCAAA | Cpf1 |
| (SEQ ID NO: 9) | sequencing |
| | primer |
| TTAAGCTGGCACTGGAGGAG | Cpf1 |
| (SEQ ID NO: 10) | sequencing |
| | primer |
| CAACATCGAGGATTGCAGGA | Cpf1 |
| (SEQ ID NO: 11) | sequencing |
| | primer |
| CACATCCTGAGCATTGACCG | Cpf1 |
| (SEQ ID NO: 12) | sequencing |
| | primer |
| ACAAGAACTTCGGCGACAAG | Cpf1 |
| (SEQ ID NO: 13) | sequencing |
| | primer |
| AGGTTATCGCTAAGTGCCAGCA | 5′ donor |
| CAGTAGTCCGTCACGCAGTAAC | Oligo- |
| AGCGACGCGIAA | nucleotide |
| AA GCGAc TCGGCTGT | with |
| ACGAg TCGCTTTT aCG C | molecular |
| GTCGCTGTTACT (SEQ ID NO: 14) | beacon target |
| | site (FIG. 2) |
| ctggagGCGTGACGGACTA | Molecular |
| CT ctccag (SEQ ID NO: 15) | beacon with |
| | 5′ 6-FAM™ |
| | and 3′ Iowa |
| | Black® |
| | (FIG. 2) |
| CTTGCATCCGGCAACTAACTTTGGA | Synthetic |
| TAATGCCCGTTTTCAGAACACGAAA | oligo |
| TTTGAACAACGTGGTCATCGTCTTG | ligated |
| GTCACGGAGTAT 2GGG | to cleaved |
| (SEQ ID NO: 16) | product |
| | (FIG. 2) |
| ACTGGTCGTCTCAGCACCTTGCATCCG | Forward |
| GC AACTAACT(SEQ ID NO: 17) | primer used |
| | to amplify |
| | ligated |
| | product |
| | (FIG. 2) |
| GACACTCGTCTCGAAACGCGTCG | Reverse |
| CTGTTACTGCGT(SEQ ID NO: 18) | primer used |
| | to amplify |
| | ligated |
| | product |
| | (FIG. 2) |
| catcgatttattatgacaac | Plasmid |
| ttgacggctacatcattcac | used |
| tttttcttcacaaccggcac | for |
| ggaactcgctcgggctggcc | Golden |
| ccggtgcattttttaaatac | Gate |
| ccgcgagaaatagagttgat | assembly |
| cgtcaaaaccaacattgcga | with PCT of |
| ccgacggtggcgataggcat | ligated |
| ccgggtggtgctcaaaagca | product |
| gcttcgcctggctgatacgt | (FIG. 2) |
| tggtcctcgcgccagcttaa | |
| gacgctaatccctaactgct | |
| ggcggaaaagatgtgacaga | |
| cgcgacggcgacaagcaaac | |
| atgctgtgcgacgctggcga | |
| tatcaaaattgctgtctgcc | |
| aggtgatcgctgatgtactg | |
| acaagcctcgcgtacccgat | |
| tatccatcggtggatggagc | |
| gactcgttaatcgcttccat | |
| gcgccgcagtaacaattgct | |
| caagcagatttatcgccagc | |
| agctccgaatagcgcccttc | |
| cccttgcccggcgttaatga | |
| tttgcccaaacaggtcgctg | |
| aaatgcggctggtgcgcttc | |
| atccgggcgaaagaaccccg | |
| tattggcaaatattgacggc | |
| cagttaagccattcatgcca | |
| gtaggcgcgcggacgaaagt | |
| aaacccactggtgataccat | |
| tcgcgagcctccggatgacg | |
| accgtagtgatgaatctctc | |
| ctggcgggaacagcaaaata | |
| tcacccggtcggcaaacaaa | |
| ttctcgtccctgatttttca | |
| ccaccccctgaccgcgaatg | |
| gtgagattgagaatataacc | |
| tttcattcccagcggtcggt | |
| cgataaaaaaatcgagataa | |
| ccgttggcctcaatcggcgt | |
| taaacccgccaccagatggg | |
| cattaaacgagtatcccggc | |
| agcaggggatcattttgcgc | |
| ttcagccatacttttcatac | |
| tcccgccattcagagaagaa | |
| accaattgtccatattgcat | |
| cagacattgccgtcactgcg | |
| tcttttactggctcttctcg | |
| ctaaccaaaccggtaacccc | |
| gcttattaaaagcattctgt | |
| aacaaagcgggaccaaagcc | |
| atgacaaaaacgcgtaacaa | |
| aagtgtctataatcacggca | |
| gaaaagtccacattgattat | |
| ttgcacggcgtcacactttg | |
| ctatgccatagcatttttat | |
| ccataagattagcggatcct | |
| acctgacgctttttatcgca | |
| actctctactgtttctccat | |
| acccgtttttttgggaattc | |
| gagctctaaggaggttataa | |
| aaaatggatattaatactga | |
| aactgagatcaagcaaaagc | |
| attcactaaccccctttcct | |
| gttttcctaatcagcccggc | |
| atttcgcgggcgatattttc | |
| acagctatttcaggagttca | |
| gccatgaacgcttattacat | |
| tcaggatcgtcttgaggctc | |
| agagctgggcgcgtcactac | |
| cagcagctcgcccgtgaaga | |
| gaaagaggcagaactggcag | |
| acgacatggaaaaaggcctg | |
| ccccagcacctgtttgaatc | |
| gctatgcatcgatcatttgc | |
| aacgccacggggccagcaaa | |
| aaatccattacccgtgcgtt | |
| tgatgacgatgttgagtttc | |
| aggagcgcatggcagaacac | |
| atccggtacatggttgaaac | |
| cattgctcaccaccaggttg | |
| atattgattcagaggtataa | |
| aacgaatgagtactgcactc | |
| gcaacgctggctgggaagct | |
| ggctgaacgtgtcggcatgg | |
| attctgtcgacccacaggaa | |
| ctgatcaccactcttcgcca | |
| gacggcatttaaaggtgatg | |
| ccagcgatgcgcagttcatc | |
| gcattactgatcgttgccaa | |
| ccagtacggccttaatccgt | |
| ggacgaaagaaatttacgcc | |
| tttcctgataagcagaatgg | |
| catcgttccggtggtgggcg | |
| ttgatggctggtcccgcatc | |
| atcaatgaaaaccagcagtt | |
| tgatggcatggactttgagc | |
| aggacaatgaatcctgtaca | |
| tgccggatttaccgcaagga | |
| ccgtaatcatccgatctgcg | |
| ttaccgaatggatggatgaa | |
| tgccgccgcgaaccattcaa | |
| aactcgcgaaggcagagaaa | |
| tcacggggccgtggcagtcg | |
| catcccaaacggatgttacg | |
| tcataaagccatgattcagt | |
| gtgcccgtctggccttcgga | |
| tttgctggtatctatgacaa | |
| ggatgaagccgagcgcattg | |
| tcgaaaatactgcatacact | |
| gcagaacgtcagccggaacg | |
| cgacatcactccggttaacg | |
| atgaaaccatgcaggagatt | |
| aacactctgctgatcgccct | |
| ggataaaacatgggatgacg | |
| acttattgccgctctgttcc | |
| cagatatttcgccgcgacat | |
| tcgtgcatcgtcagaactga | |
| cacaggccgaagcagtaaaa | |
| gctcttggattcctgaaaca | |
| gaaagccgcagagcagaagg | |
| tggcagcatgacaccggaca | |
| ttatcctgcagcgtaccggg | |
| atcgatgtgagagctgtcga | |
| acagggggatgatgcgtggc | |
| acaaattacggctcggcgtc | |
| atcaccgcttcagaagttca | |
| caacgtgatagcaaaacccc | |
| gctccggaaagaagtggcct | |
| gacatgaaaatgtcctactt | |
| ccacaccctgcttgctgagg | |
| tttgcaccggtgtggctccg | |
| gaagttaacgctaaagcact | |
| ggcctggggaaaacagtacg | |
| agaacgacgccagaaccctg | |
| tttgaattcacttccggcgt | |
| gaatgttactgaatccccga | |
| tcatctatcgcgacgaaagt | |
| atgcgtaccgcctgctctcc | |
| cgatggtttatgcagtgacg | |
| gcaacggccttgaactgaaa | |
| tgcccgtttacctcccggga | |
| tttcatgaagttccggctcg | |
| gtggtttcgaggccataaag | |
| tcagcttacatggcccaggt | |
| gcagtacagcatgtgggtga | |
| cgcgaaaaaatgcctggtac | |
| tttgccaactatgacccgcg | |
| tatgaagcgtgaaggcctgc | |
| attatgtcgtgattgagcgg | |
| gatgaaaagtacatggcgag | |
| ttttgacgagatcgtgccgg | |
| agttcatcgaaaaaatggac | |
| gaggcactggctgaaattgg | |
| ttttgtatttggggagcaat | |
| ggcgatgacgcatcctcacg | |
| ataatatccgggtaggcgca | |
| atcactttcgtctactccgt | |
| tacaaagcgaggctgggtat | |
| ttcccggcctttctgttatc | |
| cgaaatccactgaaagcaca | |
| gcggctggctgaggagataa | |
| ataataaacgaggggctgta | |
| tgcacaaagcatcttctgtt | |
| gagttaagaacgagtatcga | |
| gatggcacatagccttgctc | |
| aaattggaatcaggtttgtg | |
| ccaataccagtagaaacaga | |
| cgaagaatccatgggtatgg | |
| acagttttccctttgatatg | |
| taacggtgaacagttgttct | |
| acttttgtttgttagtcttg | |
| atgcttcactgatagataca | |
| agagccataagaacctcaga | |
| tccttccgtatttagccagt | |
| atgttctctagtgtggttcg | |
| ttgtttttgcgtgagccatg | |
| agaacgaaccattgagatca | |
| tacttactttgcatgtcact | |
| caaaaattttgcctcaaaac | |
| tggtgagctgaatttttgca | |
| gttaaagcatcgtgtagtgt | |
| ttttcttagtccgttacgta | |
| ggtaggaatctgatgtaatg | |
| gttgttggtattttgtcacc | |
| attcatttttatctggttgt | |
| tctcaagttcggttacgaga | |
| tccatttgtctatctagttc | |
| aacttggaaaatcaacgtat | |
| cagtcgggcggcctcgctta | |
| tcaaccaccaatttcatatt | |
| gctgtaagtgtttaaatctt | |
| tacttattggtttcaaaacc | |
| cattggttaagccttttaaa | |
| ctcatggtagttattttcaa | |
| gcattaacatgaacttaaat | |
| tcatcaaggctaatctctat | |
| atttgccttgtgagttttct | |
| tttgtgttagttcttttaat | |
| aaccactcataaatcctcat | |
| agagtatttgttttcaaaag | |
| acttaacatgttccagatta | |
| tattttatgaatttttttaa | |
| ctggaaaagataaggcaata | |
| tctcttcactaaaaactaat | |
| tctaatttttcgcttgagaa | |
| cttggcatagtttgtccact | |
| ggaaaatctcaaagccttta | |
| accaaaggattcctgatttc | |
| cacagttctcgtcatagctc | |
| tctggttgctttagctaact | |
| acaccataagcattttccct | |
| actgatgttcatcatctgag | |
| cgtattggttataagtgaac | |
| gataccgtccgttctttcct | |
| tgtagggttttcaatcgtgg | |
| ggttgagtagtgccacacag | |
| cataaaattagcttggtttc | |
| atgctccgttaagtcatagc | |
| gactaatcgctagttcattt | |
| gctttgaaaacaactaattc | |
| agacatacatctcaattggt | |
| ctaggtgattttaatcacta | |
| taccaattgagatgggctag | |
| tcaatgataattactagtcc | |
| ttttcctttgagttgtgggt | |
| atctgtaaattctgctagac | |
| ctttgctggaaaacttgtaa | |
| attctgctagaccctctgta | |
| aattccgctagacctttgtg | |
| tgttttttttgtttatattc | |
| aagtggttataatttataga | |
| ataaagaaagaataaaaaaa | |
| gataaaaagaatagatccca | |
| gccctgtgtataactcacta | |
| ctttagtcagttccgcagta | |
| ttacaaaaggatgtcgcaaa | |
| cgctgtttgctcctctacaa | |
| aacagaccttaaaaccctaa | |
| aggcttaagtagcaccctcg | |
| caagctcggttgcggccgca | |
| atcgggcaaatcgctgaata | |
| ttccttttgtctccgaccat | |
| caggcacctgagtcgctgtc | |
| tttttcgtgacattcagttc | |
| gctgcgctcacggctctggc | |
| agtgaatgggggtaaatggc | |
| actacaggcgccttttatgg | |
| attcatgcaaggaaactacc | |
| cataatacaagaaaagcccg | |
| tcacgggcttctcagggcgt | |
| tttatggcgggtctgctatg | |
| tggtgctatctgactttttg | |
| ctgttcagcagttcctgccc | |
| tctgattttccagtctgacc | |
| acttcggattatcccgtgac | |
| aggtcattcagactggctaa | |
| tgcacccagtaaggcagcgg | |
| tatcatcaacggggtctgac | |
| gctcagtggaacgaaaactc | |
| acgttaagggattttggtca | |
| tgagattatcA GCTTTCGCT | |
| AAggatgatttCTGGAA TTC | |
| TTCCCTATCAGTGATAGAGA | |
| TTGACATCCCTATCagtgat | |
| agagatactgagcacCGAGA | |
| CGcttcgaCGTCTCAgtttt | |
| agagctagaaatagcaagtt | |
| aaaataaggctagtccgtta | |
| tcaacttgaaaaagtggcac | |
| cgagtcggtgctttttttga | |
| agcttgggcccgaacaaaaa | |
| ctcatctcagaagaggatct | |
| gaatagcgccgtcgaccatc | |
| atcatcatcatcattgagtt | |
| taaacggtctccagcttggc | |
| tgttttggcggatgagagaa | |
| gattttcagcctgatacaga | |
| ttaaatcagaacgcagaagc | |
| ggtctgataaaacagaattt | |
| gcctggcggcagtagcgcgg | |
| tggtcccacctgaccccatg | |
| ccgaactcagaagtgaaacg | |
| ccgtagcgccgatggtagtg | |
| tggggtctccccatgcgaga | |
| gtagggaactgccaggcatc | |
| aaataaaacgaaaggctcag | |
| tcgaaagactgggcctttcg | |
| ttttatctgttgtttgtcgg | |
| tgaactggatccttaCTCGA | |
| GTCTAGACTGCAGGCggatc | |
| ttcacctagatccttttaaa | |
| ttaaaaatgaagttttaaat | |
| caatctaaagtatatatgag | |
| taaacttggtctgacaggac | |
| attatttgccgactaccttg | |
| gtgatctcgcctttcacgta | |
| gtggacaaattcttccaact | |
| gatctgcgcgcgaggccaag | |
| cgatcttcttcttgtccaag | |
| ataagcctgtctagcttcaa | |
| gtatgacgggctgatactgg | |
| gccggcaggcgctccattgc | |
| ccagtcggcagcgacatcct | |
| tcggcgcgattttgccggtt | |
| actgcgctgtaccaaatgcg | |
| ggacaacgtaagcactacat | |
| ttcgctcatcgccagcccag | |
| tcgggcggcgagttccatag | |
| cgttaaggtttcatttagcg | |
| cctcaaatagatcctgttca | |
| ggaaccggatcaaagagttc | |
| ctccgccgctggacctacca | |
| aggcaacgctatgttctctt | |
| gcttttgtcagcaagatagc | |
| cagatcaatgtcgatcgtgg | |
| ctggctcgaagatacctgca | |
| agaatgtcattgcgctgcca | |
| ttctccaaattgcagttcgc | |
| gcttagctggataacgccac | |
| ggaatgatgtcgtcgtgcac | |
| aacaatggtgacttctacag | |
| cgcggagaatctcgctctct | |
| ccaggggaagccgaagtttc | |
| caaaaggtcgttgatcaaag | |
| ctcgccgcgttgtttcatca | |
| agccttacggtcaccgtaac | |
| cagcaaatcaatatcactgt | |
| gtggcttcaggccgccatcc | |
| actgcggagccgtacaaatg | |
| tacggccagcaacgtcggtt | |
| cgagatggcgctcgatgacg | |
| ccaactacctctgatagttg | |
| agtcgatacttcggcgatca | |
| ccgcttccctcatactcttc | |
| ctttttcaatattattgaag | |
| catttatcagggttattgtc | |
| tcatgagcggatacatattt | |
| gaatgtatttagaaaaataa | |
| acaaatagctagctcactcg | |
| gtcgctacactcttcctttt | |
| tcaatattattgaagcattt | |
| atcagggttattgtctcatg | |
| agcggatacatatttgaatg | |
| tatttagaaaaataaacaaa | |
| taggggttccgcgcacattt | |
| ccccgaaaagtgccacctg | |
| (SEQ ID NO: 19) | |
| ttctcagggcgttttatggc | For |
| (SEQ ID NO: 20) | sequencing |
| | SEQ ID NO: |
| | 19 |

One skilled in the art will readily appreciate that the present disclosure is well adapted to carry out the objects and obtain the ends and advantages mentioned, as well as those inherent therein. The present disclosure described herein are presently representative of preferred aspects, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A donor oligonucleotide comprising:
a partially double stranded sequence formed by a hairpin loop;
at least a six nucleotide base overhang at the 5′ end of the oligonucleotide;
a blocked 3′ terminus;
a sequence that is a protospacer adjacent motif;
a sequence that is a RNA guided nuclease binding site;
a nuclease cleavage site at least 1 base from the 5′terminus of the oligonucleotide;
wherein the oligonucleotide is characterized by a melting temperature greater than 65° C.

2. The donor oligonucleotide of claim 1 further comprising at the 5′ terminus at least one nucleotide, N, of a target DNA sequence to be synthesized.

3. A plurality of donor oligonucleotides of claim 2, each with a unique 5′ terminus nucleotide or nucleotide subsequence, N, of a target DNA to be synthesized.

4. The donor oligonucleotide of claim 2 complexed with a class II CRISPR/Cas Cpfl nuclease and a gRNA at the protospacer adjacent motif and nuclease binding site of the oligonucleotide.

5. The complex of claim 4 wherein the donor oligonucleotide, guide RNA or nuclease are modified with a purification tag.

6. The complex of claim 5, wherein the donor oligonucleotide, guide RNA or nuclease is biotinylated.

7. An acceptor oligonucleotide comprising:
a partially double stranded sequence formed by a hairpin loop;
at least a one nucleotide base overhang at the 3′ terminus of the oligonucleotide;
a sequence that is a protospacer adjacent motif;
a sequence that is a RNA guided nuclease binding site;
a nuclease cleavage site at least one base from the 3′ terminus of the oligonucleotide;
wherein the oligonucleotide is characterized by a melting temperature greater than 65° C.

8. The acceptor oligonucleotide of claim 7 further comprising at the 3′ terminus at least one nucleotide, N, of a target DNA sequence to be synthesized.

9. A plurality of acceptor oligonucleotides of claim 8, each with a unique 3′ terminus nucleotide or nucleotide subsequence, N, of a target DNA to be synthesized.

10. The acceptor oligonucleotide of claim 8 complexed with a class II CRISPR/Cas Cpfl nuclease and a gRNA at the protospacer adjacent motif and nuclease binding site of the oligonucleotide.

11. The complex of claim 10 wherein the acceptor oligonucleotide, guide RNA or nuclease are modified with a purification tag.

12. The complex of claim 11, wherein the donor oligonucleotide, guide RNA or nuclease is biotinylated.

13. A method of synthesizing a single stranded target DNA, the method comprising the steps of:
providing a plurality of donor and acceptor oligonucleotides including:
donor oligonucleotides,
donor oligonucleotides each with unique nucleotide, or a subsequence of the target DNA sequence to be synthesized covalently bound to the 5′ terminus,
acceptor oligonucleotides, and
acceptor nucleotides, each with unique nucleotide, or subsequence of the target DNA sequence to be synthesized covalently bound to the 3′ terminus;
determining a starting point and order of addition of nucleotides necessary to form a complete target single stranded DNA sequence to be synthesized;
ligating the 5′ terminus of a donor oligonucleotide comprising N, a nucleotide or nucleotide subsequence determined to be the starting point, to the 3′ terminus of an acceptor oligonucleotide to create a ligated product;
contacting the ligated product with a guide RNA directed nuclease, to cleave the donor oligonucleotide leaving the N originating from the donor nucleotide covalently linked to the 3′ terminus of the acceptor nucleotide, thus producing an extended acceptor oligonucleotide;
purifying the extended acceptor oligonucleotide;
contacting the extended acceptor oligonucleotide, containing N, with an additional donor oligonucleotide; and
repeating ligating, cleaving and purifying steps repeatedly, extending the subsequence N with each cycle, to obtain in the final step a complete single stranded target DNA.


SEQUENCE LISTING (20 sequences)

SEQ ID NO: 1 - protein, 1297 aa, Artificial Sequence
  Synthetic
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKQNGIELFKANSDITQIQEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEQDSDVVTTMQSFYEQIAAFKTVEEKSIKETLSLLFDDLKAQKLDLSKIYFKNDKSLTDLSQQVFQDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWQKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSWNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLMIEKDRDSARKDWKKINNIKEMKEGYLSQVVHEIAKLVIEYNAIWFEDLNFGFKRGRFKVEKQVYGKLGKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 2 - DNA, 3815 bp, Artificial Sequence
  Synthetic
atgagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcmgacagtggaggammtctattmggamctctgagtctgctgttcgatgacctgamgcccagaagctggacctgagtmgatctacttcaaaaacgatmgagtctgacagacctgtcacagcaggtgtttgatgactattccgtgattgggaccgccgtcctggagtacattacacagcagatcgctccamgmcctggatmtccctctmgamgagcaggmctgatcgctmgaamccgagmggcmmtatctgagtctggamcmttmgctggcactggaggagttcmcmgcacagggatattgacamcagtgccgctttgaggamtcctggccmcttcgcagccatccccatgatttttgatgagatcgcccagmcamgacmtctggctcagatcagtattmgtaccagmccagggcaagamgacctgctgcaggcttcagcagaagatgacgtgamgccatcmggatctgctggaccagaccmcmtctgctgcacmgctgmmtcttccatattagtcagtcagaggatmggctaatatcctggatamgacgaacacttctacctggtgttcgaggmtgttacttcgagctggcamcattgtccccctgtataacaagattaggmctacatcacacagaagccttactctgacgagmgtttamctgaacttcgaaaatagtaccctggccaacgggtgggataagmcmggagcctgacmcacagctatcctgttcatcmggatgacaagtactatctgggagtgatgmtmgaaaaacaatmgatcttcgatgacamgccattaaggagmcamggggmggatacmgaaaatcgtgtatmgctgctgcccggcgcaaatmgatgctgcctmggtgttcttcagcgccaagagtatcamttctacmcccatccgaggacatcctgcggattagamtcactcaacacatactmgmcgggagcccccagaagggatatgagamtttgagttcmcatcgaggattgcaggmgtttattgacttctacmgcagagcatctccamcaccctgaatggmggattttggcttccggttttccgacacacagagatatmctctatcgacgagttctaccgcgaggtggmmtcaggggtataagctgacttttgagaacatttctgaaagttacatcgacagcgtggtcaatcagggaaagctgtacctgttccagatctataacaaagatttttcagcatacagcmgggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgaccgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagmmtcaacaacattaaggagatgaaggmggctatctgagccaggtggtccatgaattgcaaagctggtcatcgaatacaatgccattgtggtgttcgaggatctgaacttcggctttaagagggggcgctttaaggtggaaaaacaggtctatggcaagctggcaaaatgctgatcgaaaagctgaattacctggtgtttaaagataacgagttcgacaagaccggaggcgtcctgagagcctaccagctgacagctccctttgaaactttcaagmmtgggmmcagacaggcatcatctactatgtgccagccggattcacttccaagatctgccccgtgaccggctttgtcaaccactgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccmggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataactaa

SEQ ID NO: 3 - DNA, 3761 bp, Artificial Sequence
  Synthetic
atgagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaacagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggmgtttgtgmcggggagmtaccmgcgcmgggmttmcgmtacatcmtctgtatagccagcagatcmcgacaaaactctgmgamtacmgatgtctgtgctgttcamcagatcctgagtgataccgagtccmgtcttttgtcattgatamctggmgatgactcagacgtggtcactaccatgcagagcttttatgagcagatcgccgctttcmgacagtggaggmamtctattmggamctctgagtctgctgttcgatgacctgamgcccamgcgtggacctgagtmgatctacttcammcgatmgagtctgacagacctgtcacagcaggtgtttgatgactattccgtgattgggaccgccgtcctggagtacattacacagcagatcgctccamgmcctggatmtccctctmgamgagcaggmctgatcgctmgaamccgagmggcmmtatctgagtctggamcmttmgctggcactggaggagttcmcmgcacagggatattgacamcagtgccgctttgaggamtcctggccmcttcgcagccatccccatgatttttgatgagatcgcccagmcamgacmtctggctcagatcagtattmgtaccagmccagggcmgamgacctgctgcaggcttcagcagmgatgacgtgamgccatcmggatctgctggaccagaccmcmtctgctgcacmgctgmmtcttccatattagtcagtcagaggatmggctmtatcctggatamgacgmcacttctacctggtgttcgaggmtgttacttcgagctggcamcattgtccccctgtatmcmgattaggmctacatcacacagmgccttactctgacgagmgtttamctgaacttcgmmtagtaccctggccmcgggtgggatmgmcmggagcctgacmcacagctatcctgttcatcmggatgacmgtactatctgggagtgatgmtmgmamcmtmgatcttcgatgacamgccattmggagmcamggggmggatacmgamatcgtgtatmgctgctgcccggcgcamtmgatgctgcctmggtgttcttcagcgccmgagtatcamttctacmcccatccgaggacatcctgcggattagamtcactcmcacatactmgmcgggagcccccagaagggatatgagamtttgagttcmcatcgaggattgcaggmgtttattgacttctaggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgaccgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataac

SEQ ID NO: 4 - DNA, 37 bp, Artificial Sequence
  Synthetic
ctgggcaaaatgctgatcgaaaagctgaattacctgg

SEQ ID NO: 5 - DNA, 32 bp, Artificial Sequence
  Synthetic
cttgccatagacctgtttttccaccttaaagc

SEQ ID NO: 6 - DNA, 20 bp, Artificial Sequence
  Synthetic
aaggaatggtgcatgcaagg

SEQ ID NO: 7 - DNA, 20 bp, Artificial Sequence
  Synthetic
cgaatccgcctaaaacctgg

SEQ ID NO: 8 - DNA, 20 bp, Artificial Sequence
  Synthetic
attaatgccgcatcaggtcg

SEQ ID NO: 9 - DNA, 20 bp, Artificial Sequence
  Synthetic
tcctggagaacaaggccaaa

SEQ ID NO: 10 - DNA, 20 bp, Artificial Sequence
  Synthetic
ttaagctggcactggaggag

SEQ ID NO: 11 - DNA, 20 bp, Artificial Sequence
  Synthetic
caacatcgaggattgcagga

SEQ ID NO: 12 - DNA, 20 bp, Artificial Sequence
  Synthetic
cacatcctgagcattgaccg

SEQ ID NO: 13 - DNA, 20 bp, Artificial Sequence
  Synthetic
acaagaacttcggcgacaag

SEQ ID NO: 14 - DNA, 100 bp, Artificial Sequence
  Synthetic
aggttatcgctaagtgccagcacagtagtccgtcacgcagtaacagcgacgcgtaaaagcgactcggctgtacgagtcgcttttacgcgtcgctgttact

SEQ ID NO: 15 - DNA, 27 bp, Artificial Sequence
  Synthetic
ctggaggcgtgacggactactctccag

SEQ ID NO: 16 - DNA, 90 bp, Artificial Sequence
  Synthetic
cttgcatccggcaactaactttggataatgcccgttttcagaacacgaaatttgaacaacgtggtcatcgtcttggtcacggagtatggg

SEQ ID NO: 17 - DNA, 37 bp, Artificial Sequence
  Synthetic
actggtcgtctcagcaccttgcatccggcaactaact

SEQ ID NO: 18 - DNA, 35 bp, Artificial Sequence
  Synthetic
gacactcgtctcgaaacgcgtcgctgttactgcgt

SEQ ID NO: 19 - DNA, 6959 bp, Artificial Sequence
  Synthetic
catcgatttattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggaattcgagctctaaggaggttataaaaaatggatattaatactgaaactgagatcaagcaaaagcattcactaaccccctttcctgttttcctaatcagcccggcatttcgcgggcgatattttcacagctatttcaggagttcagccatgaacgcttattacattcaggatcgtcttgaggctcagagctgggcgcgtcactaccagcagctcgcccgtgaagagaaagaggcagaactggcagacgacatggaaaaaggcctgccccagcacctgtttgaatcgctatgcatcgatcatttgcaacgccacggggccagcaaaaaatccattacccgtgcgtttgatgacgatgttgagtttcaggagcgcatggcagaacacatccggtacatggttgaaaccattgctcaccaccaggttgatattgattcagaggtataaaacgaatgagtactgcactcgcaacgctggctgggaagctggctgaacgtgtcggcatggattctgtcgacccacaggaactgatcaccactcttcgccagacggcatttaaaggtgatgccagcgatgcgcagttcatcgcattactgatcgttgccaaccagtacggccttaatccgtggacgaaagaaatttacgcctttcctgataagcagaatggcatcgttccggtggtgggcgttgatggctggtcccgcatcatcaatgaaaaccagcagtttgatggcatggactttgagcaggacaatgaatcctgtacatgccggatttaccgcaaggaccgtaatcatccgatctgcgttaccgaatggatggatgaatgccgccgcgaaccattcaaaactcgcgaaggcagagaaatcacggggccgtggcagtcgcatcccaaacggatgttacgtcataaagccatgattcagtgtgcccgtctggccttcggatttgctggtatctatgacaaggatgaagccgagcgcattgtcgaaaatactgcatacactgcagaacgtcagccggaacgcgacatcactccggttaacgatgaaaccatgcaggagattaacactctgctgatcgccctggataaaacatgggatgacgacttattgccgctctgttcccagatatttcgccgcgacattcgtgcatcgtcagaactgacacaggccgaagcagtaaaagctcttggattcctgaaacagaaagccgcagagcagaaggtggcagcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatgacgcatcctcacgataatatccgggtaggcgcaatcactttcgtctactccgttacaaagcgaggctgggtatttcccggcctttctgttatccgaaatccactgaaagcacagcggctggctgaggagataaataataaacgaggggctgtatgcacaaagcatcttctgttgagttaagaacgagtatcgagatggcacatagccttgctcaaattggaatcaggtttgtgccaataccagtagaaacagacgaagaatccatgggtatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatagctctctggttgctttagctaactacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcggttgcggccgcaatcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacggggtctgacgctcagtggaacgaaaactcacgttaagggattttggtcatgagattatcagctttcgctaaggatgatttctggaattcttccctatcagtgatagagattgacatccctatcagtgatagagatactgagcaccgagacgcttcgacgtctcagttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatccttactcgagtctagactgcaggcggatcttcacctagatccttttaaattaaaaatgaagttttaaatcaatctaaagtatatatgagtaaacttggtctgacaggacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 20 - DNA, 20 bp, Artificial Sequence
  Synthetic
ttctcagggcgttttatggc